(19) United States
(12) Patent Application Publication
(10) Pub. No.: US 2003/0185963 A1
(43) Pub. Date: Oct. 2, 2003

(54) Method and apparatus for optical coating in wafer fabrication reactors

(76) Inventors: Jin Lixiang, Singapore (SG); Jianyu Martin Chen, Singapore (SG); Hwi Siong Lim, Singapore (SG); Kian Hin Victor Teo, Singapore (SG); Yee Loy Lam, Singapore (SG)

Correspondence Address: BUCKLEY, MASCHOFF, TALWALKAR, & ALLISON, 5 ELM STREET, NEW CANAAN, CT, 06840, US

(21) Appl. No.: 10/390,808

(22) Filed: Mar. 18, 2003

(30) Foreign Application Priority Data
Mar. 19, 2002 (GB) 0206482.2

(51) Int. Cl. A61B 5/103

(52) U.S. Cl. 427/1

(57) ABSTRACT

A method and a carrier frame for coating the facets of semiconductor devices, in which a plurality of bars are stacked so as to form a wafer such that the facets to be coated are exposed. The exposed facets may then be simultaneously coated in a wafer fabrication/deposition reactor to realise the associated time and cost benefits. The carrier comprises a suitable means for containing a plurality of bars and resiliently biased holding means for holding the bars in the stacked position.

### FIELD OF THE INVENTION

Laser cavities require specific optical properties such as full reflecting mirrors or semi transmitting mirrors. The construction of a small semiconductor laser diode cavity follows the same requirement for its optical performance. Depending on the desired specifications of the laser diode, several types of optical coatings may be implemented on the cavity facets. Typically, highly reflecting coatings are performed on one facet, while anti-reflection coatings are deposited on the emitting facet.

### BACKGROUND OF THE INVENTION

In the optical elements fabrication industry, such optical coatings are performed in evaporation chambers. Typically, a chamber consists of the film target boats at the bottom and the elements or components to be coated are placed at the top, as shown in FIG. 1A. The components are set into position on fixtures loaded on rotating turret hoods overhanging the film targets. An intense ion or electron beam is used to evaporate the material from the boats onto the film targets. The apparatus derives from that used to deposit optical coatings on lens and prism elements. The batch units of optical elements or prisms determine the chamber loading lots definition. Loading is frequently performed manually and very often operators swipe the optical surfaces with swabs dipped in solvents to clean the parts individually. In the event that the part or component is very small, eg several hundreds of microns in size, the implementation of the process becomes difficult.

There exists a need to apply wafer scale fabrication techniques to the manufacture of laser diode optical coatings, thereby reducing the cost of production. The primary equipment used in wafer scale fabrication lips in the planar reactors such as PECVD (Plasma Enhanced Chemical Vapour Deposition) machines. Recipes can be developed for optical coatings using these reactors. However, the ability to implement the coatings are restricted by the mandatory need for the object to be coated to mimic and emulate wafer scale topologies, mechanics and masked thermal attributes. There is a need to handle the objects or components to be coated in an exact wafer-like manner. The component loading access carriages in most wafer reactors are normally constructed with a low profile for enhanced reactor performance. This further demands wafer scale management of parts to be coated in wafer reactors.

Regarding the physical laser facet coatings, most laser diodes are cleaved in the lattice planes to create the laser diode cavity construction. From these cleaved facets, the laser light will be emitted or reflected back into the diode. The optical coatings must be deposited on the faces of the cleaved facets.

To achieve this, there is a common procedure to reduce a wafer full of diodes into bars on the diode substrate, with each bar consisting of many diodes. The laser bars are cleaved such that losing cavity forms the bar height, as shown in FIG. 1B. When the laser bars are stacked together, only both the end facets to be coated are exposed to the coating chambers. Currently, there are machines available to stack and bind the laser bars on fixtures in preparation for loading into the optical coating evaporation chambers. The exact detail of the current mode of stacking, art of fixtures and confinement of the bars is not fully known commercially in the public domain. The cost of such machines is very high.

Such a loading machine provides only intermediate batch segregation and is dedicated in populating the loading fixture. Furthermore, the cost of ownership of these niche machines is very high. The basis of the break down in manufacturing economy lies in the handling of the small components and in managing the coating of these components.

Consequently, a well-conceived design of wafer-like laser bar carrier together with a laser bar stack loader suited for use in wafer reactors would reduce the cost of coating laser diodes drastically.

### SUMMARY OF THE INVENTION

According to the present invention, a carrier for bars of semiconductor devices to be coated with a coating comprises:

means for containing a plurality of bars stacked such that the facets to be coated are exposed, and

resiliently biased holding means for holding the bars in the stacked position.

According to a second aspect of the present invention, a method of coating bars of semiconductor devices with a coating comprises the steps of:

stacking a plurality of bars of devices such that the facets to be coated are exposed;

holding the stack in position by a resiliently biased force; and

depositing a coating on the exposed facets.

According to a third aspect of the present invention, a loading apparatus for loading bars of semiconductor devices into a carrier frame comprises:

loading means for individually loading bars into the carrier frame to form a stack; and

supporting means for supporting the stack of bars which have been loaded into the carrier frame, wherein the supporting means is moveable such that, as each bar is loaded into the carrier frame, the supporting means moves to move the stack of bars to accommodate the next bar to be loaded.

In the method and carrier frame of the present invention, the laser bars are stacked in contact with each other with the facets to be coated exposed. Typically, these facets will be the smaller end facets of the bar, which in the case of a laser diode define the two ends of the laser cavity. Of course, the bars may be stacked with a different orientation so as to expose different facets of the bar for coating.

Thus, once stacked, the surfaces that are not to be coated are touching each other and are protected from the coating process. This can be particularly important in the case of laser diodes, where the top and bottom surfaces may have functional features such as metallized regions for electrical contacts. Once stacked within the carrier, the side facets of the bars may also be protected.

The overall effect is that the stack of bars can then be treated as a single wafer, with the facets to be coated forming part of the surface of the wafer. The stack is held in position by a resiliently biased force to allow for thermal expansion of the devices during the coating process. The resiliently biased force absorbs any thermal expansion of the devices. The present invention is particularly suited for applying optical coatings, but may be utilised for other coatings such as depositing protective coatings on mirror facets.

Preferably, the facets to be coated form a contiguous surface in the stacked position.

Preferably, the carrier comprises a carrier frame formed from a sheet material having a cut-out for containing the stack of bars. Preferably, the thickness of the sheet material is substantially the same as that dimension of a bar substantially perpendicular to the facet to be coated. In the case of a laser diode this will typically be the length of a bar, which determines the length of the laser cavity. Thus, when the bars are in position in the carrier, the arrangement simulates a wafer, and can be handled as such.

Preferably, the carrier frame is formed from a resilient material. Preferably, the resiliently biased holding means comprises a resilient force applied to the laser bars by a portion of the resilient carrier frame.

Preferably, the resilient force is applied by a portion of the resilient carrier plate to a clamp anvil which is stacked with the stack of bars.

Preferably, the carrier includes at least one packing slab for positioning adjacent the stack of bars. Preferably, a packing slab is positioned at the top and bottom of the stack of bars.

A preferable material for the carrier plate is sheet steel or similar material with restraint capability.

### BRIEF DESCRIPTION OF THE DRAWINGS

Examples of the present invention will now be described with reference to the accompanying drawings in which:

FIG. 1A shows an optical coating chamber;

FIG. 1B shows a prior art carrier for laser bars;

FIG. 2 shows a plan view of a carrier in accordance with a first embodiment of the invention;

FIG. 3A shows a side elevation and FIG. 3B shows a plan view of a carrier in accordance with a second embodiment of the present invention;

FIG. 4A illustrates the apparatus for assembling laser bars into a carrier and 4B shows the associated packing tools;

FIG. 5 illustrates the assembly of laser bars into the carrier; and

FIG. 6 shows the loading of the carrier frame into the reactor.

### DETAILED DESCRIPTION

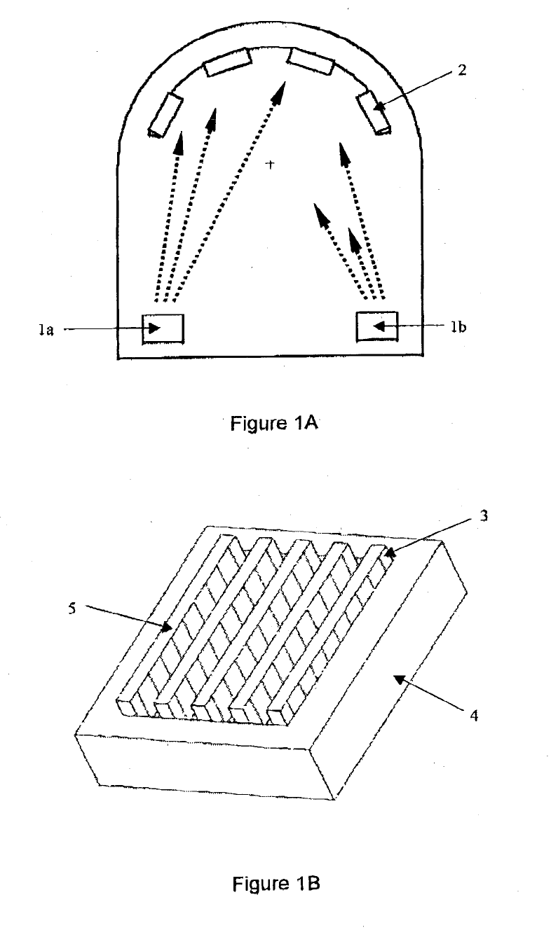

FIG. 1A shows an optical coating chamber, having target boats 1A, 1B at the bottom and the components 2 to be coated are placed at the top. An intense ion or electron beam is used to evaporate the material from the boats 1A, 1B which is then deposited onto the components 2.

FIG. 1B shows a prior art arrangement for clamping laser bars 3 in a frame 4. The laser bars 3 are clamped individually and are spaced from each other with the facets 5 for optical coating facing exposed.

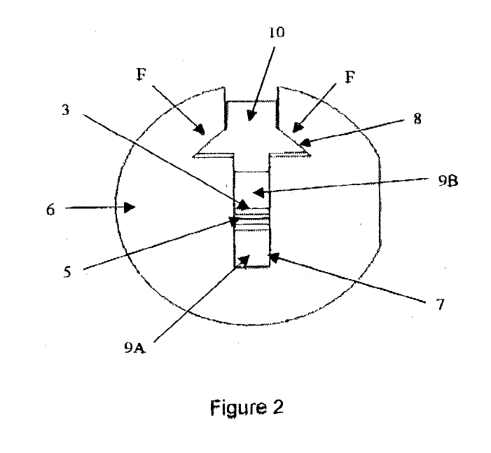

FIG. 2 shows a carrier in accordance with the present invention. A carrier comprises a carrier frame 6, which is formed from thin sheet steel. The frame 6 has a recess 7 into which a plurality of laser bars 3 are stacked touching each other with the facets 5 to be coated exposed, to emulate a wafer with a single contiguous surface formed from the adjacent facets 5.

The recess 7 has an enlarged trapezoidal section 8 at the top. The configuration of the recess may be suitably formed out of triangular or slight curvilinear shape for optimum packing. The laser bars 3 are packed into the recess 7, with packing elements 9A, 9B at the top and bottom of the stack. The packing elements 9A, 9B are made of a resilient material. A clamping anvil 10 is inserted into the enlarged section 8 of the recess 7. The clamping anvil 10 has a generally trapezoidal shape to fit into the enlarged section 8 of the recess 7, and is made from a resilient material. The preferential shape is one that allows a slight clamping force to be transmitted to packing element 9B.

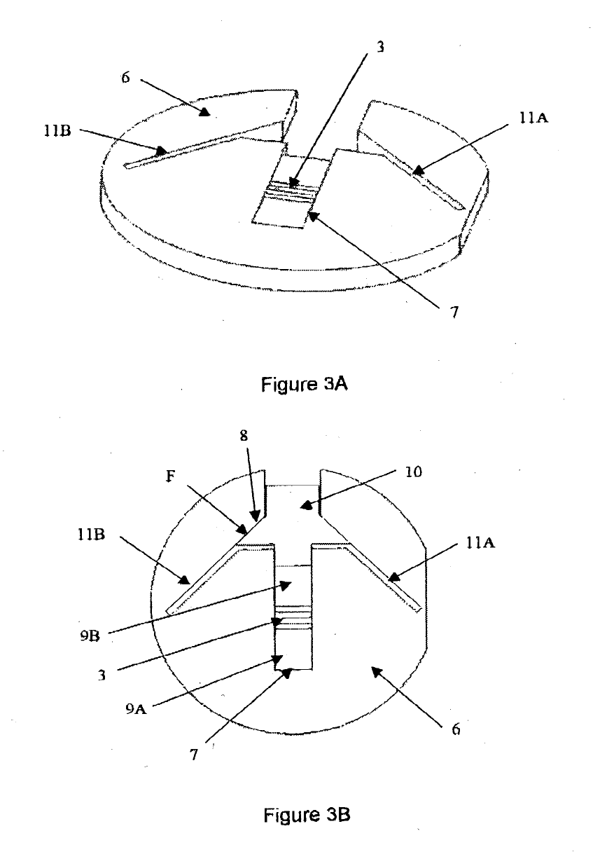

FIG. 3 shows an alternative configuration wherein the enlarged trapezoidal section 8 of the recess 7 has two Slots 11A, 11B extending outwards from it. This embodiment allows a selective force to be applied to packing element 9B. Extensions 11A and 11B are not limited to linear straight structural configuration, and preferably include shapes that allow stability and self-restraint of clamp anvil 10.

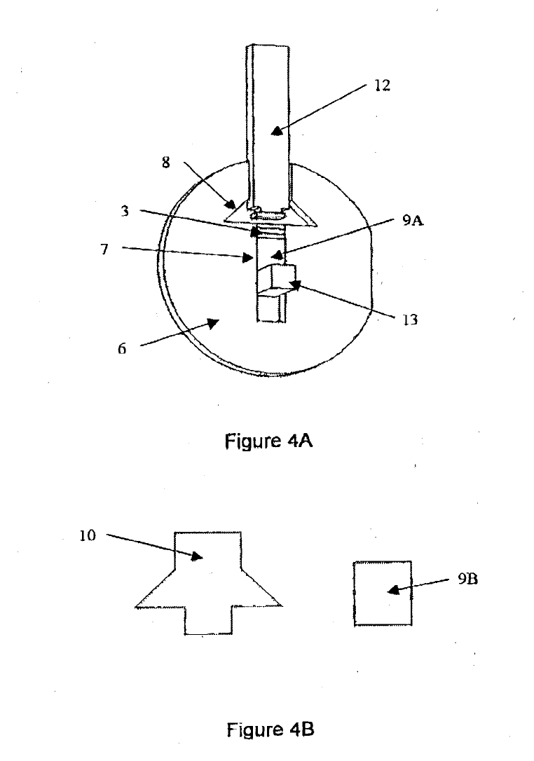

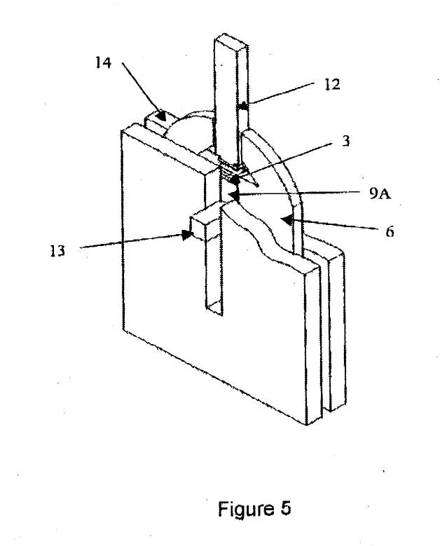

FIGS. 4 and 5 illustrate the method of assembly of the laser bars 3, the packing slabs 9A, 9B and the clamp anvil 10 into the carrier frame 6. A packing slab 9A and the laser bars 3 are loaded into the recess 7 In the carrier frame 6 by means of a loader 12, which holds each element by vacuum. A further packing plate 9B is then loaded on top of the stack of laser bars 3. The loaded elements 3, 9A, 9B are supported by a bearer pinion 13 which is moved downwards as the elements 3, 9A, 9B are loaded. The carrier frame 6 is supported by vertical backer plates 14, as shown in FIG. 5.

Once the laser bars 3 and the packing plates 9A, 9B are loaded, the anvil 10 is inserted. A strut or caliper jaw is used to slightly lever open the top of the enlarged section 8 of the recess 7 to facilitate insertion of packing anvil 10 from the side. When the anvil 10 is inserted, the top of the recess is released, and a spring force F acts on the sloping side sections of the clamp anvil 10 to resiliently bias it downwards, applying a resilient biasing force to the packing slab 9A and the stack of laser bars 3. The spring force F can be reduced by the provision of slots 11A, 11B, as shown in FIG. 3, and the force can be varied by varying the length of the slots 11A, 11B.

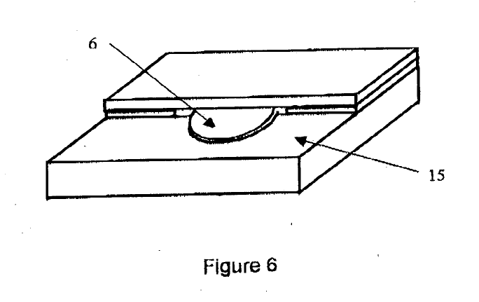

FIG. 6 illustrates the loading of the carrier frame 6 into the reactor chamber by placing the carrier frame 6 onto a carrier plate 15, which slides into the reactor. The arrangement of the present invention is such that the facets 5 of the laser bears 3, which are to be coated, form a contiguous surface that simulates a single wafer. The top and bottom surfaces of the laser bars 3 are protected from optical coating.

1. A carrier for bars of semiconductor devices to be coated with a coating comprising:
means for containing a plurality of bars stacked such that the facets to be coated are exposed; and
resiliently biased holding means for holding the bars in the stacked position.

2. A carrier according to claim 1, comprising a carrier frame formed from a sheet material having a cut-out for containing the stack of bars.

3. A carrier according to claim 2, wherein the thickness of the sheet material is substantially the same as that dimension of a bar substantially perpendicular to the facet to be coated.

4. A carrier according to claim 2 or 3, wherein the carrier frame is formed from a resilient material.

5. A carrier according to claim 4, wherein the resiliently biased holding means comprises a resilient force applied to the laser bars by a portion of the resilient carrier frame.

6. A carrier according to claim 5, including a clamp anvil to be stacked with the stack of bars, wherein the resilient force is applied by a portion of the resilient carrier plate to the clamp anvil.

7. A carrier according to any one of claims 2 to 6, including at least one packing slab for positioning adjacent the stack of bars.

8. A method of coating bars of semiconductor devices with a coating comprising the steps of:
stacking a plurality of bars of devices such that the facets to be coated are exposed;
holding the stack in position by a resiliently biased force; and
depositing a coating on the exposed facets.

9. A method according to claim 8, wherein the facets to be coated form a contiguous surface in the stacked position.

10. A loading apparatus for loading bars of semiconductor devices into a carrier frame comprising:
loading means for individually loading bars into the carrier frame to form a stack; and
supporting means for supporting the stack of bars which have been loaded into the carrier frame, wherein the supporting means is moveable such that, as each bar is loaded into the carrier frame, the supporting means moves to move the stack of bars to accommodate the next bar to be loaded.
